(19) United States
(12) Patent Application Publication
(10) Pub. No.: US 2012/0041379 A1
(43) Pub. Date: Feb. 16, 2012

(54) Cartridge and Medication Delivery Device

(75) Inventors: Ross Macarthur, Cheshire (GB); Graham Jay, Frankfurt (GB); Christopher James Smith, Cheshire (GB)

(73) Assignee: SANOFI-AVENTIS DEUTSCHLAND GMBH, Frankfurt am Main (DE)

(21) Appl. No.: 13/146,015

(22) PCT Filed: Jan. 21, 2010
(86) PCT No.: PCT/EP10/50670

§ 371 Date: Oct. 26, 2011

(30) Foreign Application Priority Data
Jan. 30, 2009 (EP) 09001270.9
Mar. 17, 2009 (US) 61160862

(51) Int. Cl. A61M 5/31; A61M 5/32; A61M 5/00

(52) U.S. Cl. 604/192; 604/93.01; 604/244; 604/200

(57) ABSTRACT

A single-use cartridge for a medication delivery device, comprises a body element (1) having a proximal end and a distal end, a dispensing means (5), and a deformable capsule (3) containing medication. The capsule (3) is disposed inside the body element (1) and has a skin which encases the medication. The capsule (3) is configured to be pushed in the distal direction against the dispensing means (4) so that the content of the capsule (3) is dispensed.

The invention relates to a single-use cartridge for a medication delivery device and the respective medication delivery device.

WO 2008/068502 shows a dosage delivery device having a housing within which is formed a reservoir, with a plunger located within the reservoir and moveable to dispense a material from a discharge opening of the reservoir. The device comprises a plunger actuation mechanism which is moveable between a contracted storage position in which the mechanism is held at least partially within the housing and an extended primed position in which the plunger can be actuated. The device additionally comprises a priming mechanism slideably mounted with respect to the housing and moveable between a storage and a primed position to expose the discharge opening and to release the plunger actuation mechanism.

DE 2719815 shows a hypodermic syringe with a needle extending from a disposable capsule containing a fluid drug. The capsule is partially rigid and partially flexible. A plunger of the syringe compresses the flexible part, thus the capsule collapses to deliver the fluid drug.

WO 95/23622 shows a predetermined dosage hypodermic syringe system for injecting a predetermined dosage of therapeutic fluid. A sealed capsule contains the fluid to be injected. A double-ended hypodermic needle includes a first end for piercing engagement with a body tissue and a second end for piercing engagement with the capsule. The capsule is disposed between a plunger and an end wall of a barrel member. Advancement of the plunger moves the capsule into the piercing engagement with the second end of the needle, collapsing the capsule and discharging the therapeutic fluid through the needle.

It is an aim of the present invention to provide alternative medication delivery means for delivery of the contents of a capsule.

For this purpose a single-use cartridge comprises a body element which has a proximal end and a distal end and dispensing means. A deformable capsule containing medication is disposed inside the body element. The capsule has a skin which encases the medication. The capsule is configured to be pushed in the distal direction towards the dispensing means before the content of the capsule is dispensed. The dispensing means is suitable to open the capsule and then the capsule can be compressed and the content can be administered to a patient by means of the dispensing means.

A preferred cartridge comprises a bung element which is moveable in the distal direction upon an external force being exerted, whereupon the capsule is pushed towards the dispensing means so that the content of the capsule is dispensed.

In one embodiment the dispensing means is configured to cut open, puncture or rupture the capsule so that the medication is dispensed. In one embodiment puncturing the capsule results in forming a hole in the skin of the capsule. In another embodiment cutting open the capsule results in forming a cut in the skin of the capsule. In an alternative embodiment the capsule is ruptured before delivering the medication by the dispensing means.

A preferred embodiment of the dispensing means comprises an injection needle having a proximal end configured to puncture the capsule. Contrary to a conventional syringe medication delivery system, there is no need to attach a needle prior to dispensing a dose, because the needle is part of the cartridge. The needle provided in the cartridge is preferably a single-use needle. Thus, reuse of the needle is not intended in order to prevent cross-contamination or infection.

In one embodiment a distal part of the body element is designed as a needle shield which the needle is disposed in for the purpose of needle protection in order to prevent patient injuries. Alternatively a needle shield is fixed to the distal part of the body element, the needle being disposed in the needle shield. From this protected position the needle can be moved in the distal direction so that a distal part of the needle extends out of the needle shield to be inserted into the skin of a patient. If this distal movement of the needle is carried out when the distal end of the needle shield is already pressed against the skin of the patient in preparation of an injection, the patient does not see the needle before injection, which is advantageous for needle-phobic patients.

One embodiment comprises a needle which can be moved in the proximal direction after moving in the distal direction so that the needle retracts into the needle shield after use. In this case the patient would not need to see the needle at any time during treatment, which is advantageous for needle-phobic patients.

In another embodiment the distal part of the body element is positioned inside the needle shield. The needle shield is moveable in the distal direction with respect to the body element. The dispensing means with the needle are located inside the body element. In one embodiment the distal end of the body element and the distal end of the needle shield are aligned or nearly aligned. This construction is compact and the needle located inside the body element is protected by the body element and needle shield before use. In a preferred embodiment the body element cannot move distally with respect to the needle shield, but the dispensing means are movable in the distal direction with respect to the body element and the needle shield. An alternative embodiment has a body element which is slightly movable in the distal direction with respect to the needle shield. Thus, the needle extends out of the body element and the needle shield, when the dispensing means is moved in the distal direction with respect to the body element. After medication delivery the dispensing means is not movable with respect with to the body element, but the body element is retractable in the proximal direction with respect to the needle shield, resulting in retracting the needle into the needle shield. In other words, the needle shield can be moved in the distal direction with respect to the body element, so that the needle is disposed in the needle shield after medication delivery.

A preferred embodiment of the cartridge is configured such that the dispensing means with the needle and the compressed deformable capsule are moved in the proximal direction after medication delivery when the bung element is moved in the proximal direction.

One embodiment of the needle shield has a distal wall comprising a seal configured to be punctured by the needle when the needle is moved in the distal direction so that the needle is not visible before injection. Furthermore, contamination of the needle is prevented and sterility of the needle is maintained by means of the seal. In one embodiment, the body element has a distal wall or a distal end which comprises a seal.

The cartridge is used in connection with a medication delivery device. One preferred embodiment of the medication delivery device is reusable. An alternative embodiment of the medication delivery device is designed as a single-use device. The medication delivery device comprises a piston element which can be moved in the distal direction. The piston element is configured to push the bung element of the cartridge in the distal direction so that the bung pushes the capsule towards the dispensing means which may result in distally pushing the dispensing means.

One embodiment of the cartridge comprises first coupling means which can be releasably coupled with the piston element of a medication delivery device so that the needle is drawn back into the needle shield. In one embodiment the bung of the cartridge comprises the first coupling means. After medication delivery the bung element is attached to the dispensing means, e.g. by engaging means, so that retracting the bung results in retracting the dispensing means with the needle in the proximal direction. In an alternative embodiment the body element of the cartridge which has a moveable needle shield comprises the first coupling means. When the piston element is retracted the body element is proximally moved with respect to the needle shield, resulting in drawing back the needle into the needle shield.

An alternative embodiment of the cartridge comprises dispensing means configured to deliver the medication in a jet spraying manner. The cartridge is configured to deliver the medication needle-less into the human body by using a high-pressure jet of the liquid medication instead of a hypodermic needle to penetrate the patient's epidermis.

An alternative embodiment of the cartridge is configured to distribute the medication over the skin of the human body.

An embodiment of a medication delivery device can be releasably coupled with the cartridge. The medication delivery device is configured to move the capsule and/or the bung element in the distal direction.

An embodiment of the medication delivery device comprises a piston element moveable in the axial direction. The piston element is suitable to move the bung element in the distal direction for medication delivery and may also be suitable to move the bung element and/or the body element in the proximal direction after medication delivery.

A preferred medication delivery device comprises obstructions configured to stop or prevent a proximal movement of the cartridge when the piston element is moved in the proximal direction after medication delivery so that the piston element is detached from the cartridge before the cartridge is removed from the medication delivery device.

Other features will become apparent from the following detailed description when considered in conjunction with the accompanying drawings.

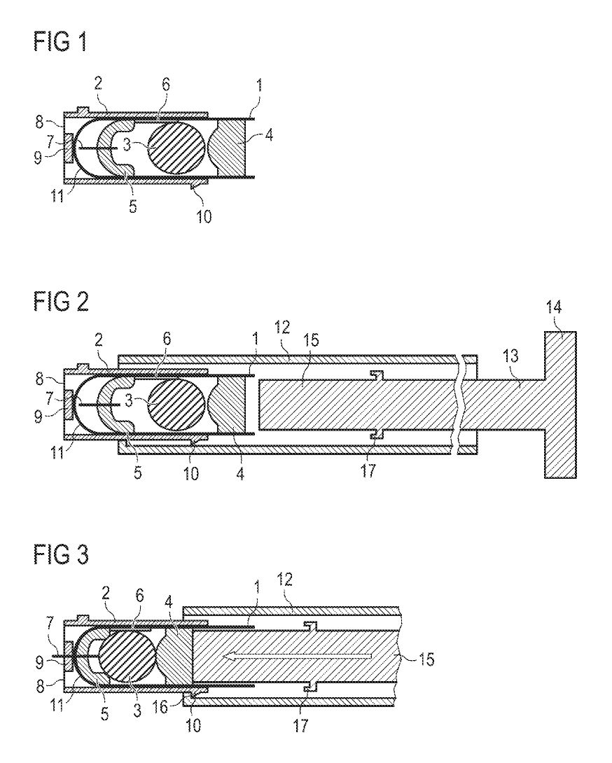

FIG. 1 shows an embodiment of a cartridge.

FIG. 2 shows the cartridge according to FIG. 1 inserted into an embodiment of a medication delivery device.

FIGS. 3 to 8 show stepwise the operation of the cartridge and the medication delivery device.

FIG. 9 shows an alternative embodiment of the cartridge.

FIG. 1 shows an embodiment of a cartridge. The cartridge comprises a body element 1 having a proximal end and a distal end. One embodiment of the body element 1 is formed cylindrically. The body element 1 can be made of glass. Preferably, the body element 1 is made of a material other than glass, e.g. of plastic, which is beneficial because breakage of glass in mass production is an issue.

A needle shield 2 is located at the distal part of the body element 1. A first embodiment of the needle shield 2 can be moved in the distal direction relative to the body element 1 and the elements disposed inside the body element 1. A second embodiment of the needle shield 2 (not shown) is fixed to the body element 1. Alternatively the distal part of the body element 1 is formed as needle shield 2 (not shown).

The cartridge comprises dispensing means 5 located inside the body element 1. The dispensing means 5 is suitable to open the skin of the capsule 3 and to administer the content of the capsule 3 to a patient. For this purpose the dispensing means 5 comprises an injection needle 7 suitable to inject medication. The dispensing means 5 and the needle 7 form a needle assembly.

The dispensing means 5 is located within the body element 1 such that the needle 7 is disposed inside the body element 1. The needle shield 2 surrounds the needle 7 containing distal part of the body element for the purpose of needle protection for the user's safety. In an initial position the distal part of the body element 1 is positioned inside the needle shield 2. In a preferred embodiment the distal end of the body element is aligned with the distal end of the needle shield. The needle assembly 5, 7 is located inside the body element so that the needle 7 does not extend from the distal end of the body element 1. The first embodiment of the needle shield 2 is moveable only in the distal direction with reference to the body element 1.

The dispensing means 5 can be moved in the distal direction relative to the body element 1 so that the needle 7 extends from the distal end of the needle shield 2 before injection. The needle 7 can be moved into an extended position with reference to the needle shield 2 by moving the dispensing means 5 in the distal direction. In the extended position the needle 7 extends from the distal ends of the body element 5 and the needle shield 2.

When the body element 1 is moved in the proximal direction with reference to the needle shield 2, the extended needle 7 and the dispersing means 5 are retracted in the proximal direction with reference to the needle shield 2.

“Retractable” or “retract” shall mean that the respective element is configured to be moved in the proximal direction. If the respective element has been moved in a distal direction before retraction the respective element is not necessarily to be retracted to its initial position.

In case of the second embodiment of the needle shield 2, which is fixed to the body element 1, the dispensing means 5 can be moved in the axial direction relative to the body element 1 and the needle shield 2. “Moveable in the axial direction” means that the dispensing means 5 can be moved in the distal direction and retracted in the proximal direction. The dispensing means 5 can be moved in the distal direction relative to the needle shield 2 so that the needle 7 extends from the distal end of the needle shield 2 before injection. The dispensing means 5 is retractable in the proximal direction with respect to the body element 1 after moving in the distal direction so that the needle 7 retracts into the needle shield 2 after injection. In one embodiment the dispensing means 5 can only be moved in the distal direction with respect to the body element 1.

An alternative embodiment (not shown) of the cartridge without a needle shield has immoveable dispensing means. In this embodiment the needle is neither extracted before injection nor retracted after injection. In other words, the needle is permanently located at the distal end of the cartridge.

A block element 11 is located at the distal part of the body element 1; the block element 11 is configured to stop the distal movement of the dispensing means 5. The block element 11 has an opening for the distally moving needle 7. In this embodiment the form of the block element 11 is designed to match with the form of the dispensing means 5. The proximal side of the block element 11 is formed like the distal side of the dispensing means 5.

A distal wall 8 of the needle shield 2 comprises a pierceable seal 9 arranged such that the needle 7 pierces the seal 9 when the needle 7 moves in the distal direction.

The cartridge further comprises a bung element 4 disposed in a proximal part of the body element 1. The bung element 4 can be moved in the distal direction upon being pressed by external force. In one embodiment the bung element 4 is retractable in the proximal direction by an external force. The retractable bung element 4 is preferably suitable to retract the dispensing means 5 and the needle 7 of the cartridge, thereby moving the needle 7 into a safe position within the needle shield 2 and/or body element 1.

A deformable capsule 3 containing medication is located inside a chamber formed by the body element 1, the dispensing means 5 and the bung element 4. The capsule 3 has a flexible skin encasing the medication, which is preferably a gel medication or a liquid medication. The capsule 3 contains a single dose of the medication to be dispensed. The single dose capsule 3 is also named “primary package”. In one embodiment the capsule 3 is formed as a ball. In an alternative embodiment the capsule is formed like an ellipsoid. One embodiment of the capsule is similar to the soft gelatine type which may be used for vitamins. Different sizes of primary packages can be used for different doses. Alternatively the fill level or concentration of the medication within the primary package can be varied. Thus, it is simple to vary the dose amount or strength during manufacturing of the cartridges.

In this embodiment the size of the capsule 3 corresponds to the cross-section of the body element 1. The position of the capsule 3 is fixed within the body element 1 due to friction. In an alternative embodiment the capsule 3 is adhered to the body element 1. In another alternative embodiment the capsule 3 is not fixed inside the body element 1 at all.

A gas exhaust 6 is configured such that gas, e.g. air, in the chamber between the capsule 3 and the dispensing means 5 escapes when the capsule 3 is moved towards the dispensing means 5.

The cartridge comprises first stopping means 10 arranged on the outside of the needle shield 2. The first stopping means 10 is configured to stop or obstruct axial movement of the cartridge in a medication delivery device. The movement of the cartridge to an axial direction is stopped by the first stopping means 10 if the body element 1 cannot move to the axial direction with respect to the needle unit 2.

The body element 1 of the cartridge may or may not be labelled with additional instructions, warnings or explanatory messages.

An alternative embodiment of the cartridge (not shown) without a bung element comprises a proximal wall of the body element. The proximal wall is configured to be impressed by e.g. a piston of a medication delivery device which directly pushes the capsule in the distal direction towards the dispensing means so that the content of the capsule can be dispensed. The proximal wall is not necessary if the capsule is fixed or adhered inside the body element so that the capsule does not fall out of the body element and so that the sterility of the proximal end of needle 7 is maintained.

Embodiments of cartridges having an integrated needle assembly, as described above, can e.g. be used in a reusable auto injector without the need for needle attachment.

FIG. 2 shows the embodiment of the cartridge according to FIG. 1, the cartridge being inserted into an embodiment of a medication delivery device. The medication delivery device is suitable to drive the bung element 4 in order to puncture the capsule 3 so that the medication can be delivered.

The medication delivery device comprises a housing 12. A piston element 15 is at least partly located inside the housing 12 and can be moved in the distal direction e.g. manually or automatically by means of a spring or a motor. The piston element 15 is configured to push the bung element 4 of the cartridge in the distal direction.

The medication delivery device according to FIG. 2 further comprises a drive element 13 having a button part 14 configured to be pushed by a user in the distal direction and to be pulled by the user in the proximal direction after pushing. The drive element 13 is coupled (e.g. directly or via a gear element) with the piston element 15 so that the piston element 15 moves in the distal direction when the drive element 13 is pushed in the distal direction. The piston element 15 retracts in the proximal direction when the drive element 13 is pulled in the proximal direction. In one embodiment the piston element 15 and the drive element 13 are coupled or formed as one piece.

In a preferred embodiment the piston element 15 is pushed in the distal direction and pulled in the proximal direction automatically by the delivery device, e.g. by a spring or motor, without requiring a force input from the user. Thus, once the delivery device is triggered by the user the insertion of the needle, delivery of the injection and retraction of the needle will all occur without further user action.

For delivering a dose of medication, the cartridge is (preferably releasably) coupled to the distal part of the housing 12. In the embodiment shown in FIG. 2 the cartridge is inserted into the distal part of the housing 12. In an alternative embodiment (not shown) the cartridge is releasably coupled to the housing 12 only with its proximal end, e.g. by means of a thread.

In a preferred embodiment each cartridge is individually supplied to the medication delivery device. In other words, one cartridge is supplied to the medication delivery device by the user, the medication is delivered and the cartridge is removed by the user; then the next cartridge is supplied by the user. In an alternative embodiment a magazine of multiple cartridges is supplied to the medication delivery device. Removing the used cartridge and supplying the next cartridge is performed automatically or semi-automatically. If the cartridges are provided in a sealed magazine, the used cartridges clearly indicate if the dose has been administered or how many doses have been administered.

If the cartridge is not made of glass, the design of the device will no longer need to be adapted to a standard glass cartridge or syringe containing the medication. Thus, embodiments of the medication delivery device can be smaller, more discrete, more stylish and cheaper than conventional devices.

FIGS. 3 to 8 show the operation of the cartridge and the medication delivery device by displaying the distal part of the arrangement comprising the cartridge and part of the medication delivery device during consecutive operation steps.

FIG. 3 illustrates that the piston element 15 moves in the distal direction when the button element 14 is pushed in the distal direction. After the piston element 15 has reached the bung element 4 at the proximal end of the cartridge the cartridge is moved in the distal direction when the piston element 15 further moves in the distal direction.

The distal movement of the cartridge is stopped when the first stopping means 10 of the cartridge reaches a stopping edge 16 of the housing 12. The stopping edge 16 is configured to mechanically interact with the stopping means 10 so that the cartridge is prevented from moving further in the distal direction with respect to the housing 12 when the piston element 15 is pushed further in the distal direction. Due to further distal movement of the piston element 15, the bung element 4 is pushed in the distal direction with respect to the body element 1 so that the capsule 3 is pushed towards the dispensing means 5.

In one embodiment the cartridge moves in the distal direction until it is stopped by the stopping edge 16 and then the bung element 4 moves in the distal direction. To achieve this the force required to distally move bung element 4 relative to the body element 1 is greater than the force require to distally move the cartridge relative to the housing 12. In an alternative embodiment the cartridge moves in the distal direction while the bung element 4 also moves in the distal direction. In another embodiment only the bung element 4 is moved by the piston element 15 in the distal direction and the cartridge does not move at all with respect to the housing 12 after it has been correctly inserted into the housing 12.

The dispensing means 5 are pushed by the capsule 3 in the distal direction relative to the body element 1 so that the needle 7 extends from the distal end of the needle shield 2, which remains aligned with the distal end of the body element 1. The distal movement of the dispensing means 5 is stopped, when it reaches the block element 11 located at the distal end of the body element 1.

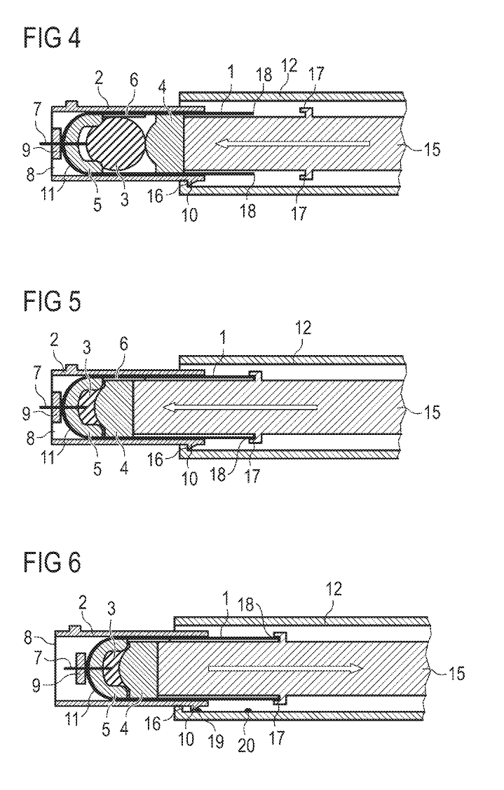

FIG. 4 shows that the capsule 3 is compressed when the capsule 3 is pushed against the dispensing means 5. The dispensing means 5 is designed to prevent piercing engagement between the capsule 3 and the proximal end of the needle 7 as long as the capsule 3 is not compressed. In one embodiment the inner cross-section of the dispensing means 5 is smaller than the one of the body element 1 and therefore also smaller than the outer cross-section of the capsule 3. This configuration prevents unintended damage of the capsule 3, e.g. when the capsule 3 moves inside the body element 1 without being pushed by the bung element 4 against the dispensing means 5. The dispensing means 5 is also designed to guide the capsule 3 so that it is punctured by the needle 7 when the capsule 3 is pushed far enough.

The bung element 4 pushes the capsule 3 towards the dispensing means 5 and then against the dispensing means 5 so that the capsule 3 is compressed. Due to the compression the capsule 3 is pushed towards the proximal end of the needle 7. The needle 7 punctures the skin of the capsule 3.

The air trapped between the capsule 3 and the dispensing means 5 escapes through the gas exhaust 6 during the distal movement of the capsule 3. One embodiment of the gas exhaust 6 is designed as a pipe. An alternative embodiment of the gas exhaust 6 is designed as a groove formed in the inside wall of the body element 1. The gas exhaust 6 extends from the dispensing means 5 in the proximal direction so that the trapped air can escape.

FIG. 5 shows that the content of the capsule 3 is dispensed through the needle 7 when the piston element 15 is further moved in the distal direction. The punctured capsule 3 is compressed by the continued force in the distal direction, resulting in dispensing the liquid content or gel content of the capsule through the needle 7 while the capsule is deformed.

The distal movement of the piston element 15 and the compression of the capsule 3 are stopped when the second stopping means 17 arranged on the piston element 15 reaches the distal end 18 of the body element 1. In an alternative embodiment of the medication delivery device (not shown) the second stopping means reaches further stopping means disposed inside the housing 12.

The above-mentioned compression of the capsule 3 is achieved by pushing the piston element 15 in the distal direction.

After medication delivery the piston element 15 can be retracted.

FIGS. 6 to 8 show the proximal movement of the piston element 15 which is releasably coupled to the cartridge. These figures relate to a cartridge having a needle shield 2 which is moveable with respect to the body element 1 according to the first embodiment of the needle shield.

In one embodiment the body element 1 is releasably coupled with the piston element 15, e.g. by snapping means (not shown). The piston element 15 and the body element 1 may be coupled when the piston element 15 reaches its furthest distal position. In one embodiment the second stopping means 17 and the proximal end of the body element 18 may be designed as engagement means (not shown) configured to engage when the second stopping means 17 reach the proximal end of the body element 18.

In an alternative embodiment the piston element 15 is releasably coupled with the bung element 4, e.g. by snapping means (not shown). After medication delivery the bung element 4 does not move or barely moves with respect to the body element 1 so that proximal movement of the bung element 4 is transferred to the body element 1. The body element 1 is attached to the bung element 4 after medication delivery due to an attachment force. In one embodiment the attachment force results from the collapsed capsule 3 located between the dispensing means 5 and the bung element 4, wherein the dispensing means 5 may be attached to the body element 1 by locking means (not shown) or friction. In another embodiment the attachment force results from locking means (not shown) between the bung element 4 and the body element 1 which engage when the bung element 4 reaches its furthest distal position. In another embodiment friction between the bung element 4 and the body element 1 provides sufficient attachment force.

The cartridge comprises first stopping means 10 located on the outside wall of the needle shield 2. The medication delivery device comprises first and second obstructions 19, 20 located on the inside wall of the housing 12. The first and second obstructions 19, 20, which can be designed as bumps or elevations, are configured to interact with the first stopping means 10.

The cartridge is rotatable relative to the housing 12. In a first positioning the cartridge is positioned relative to the housing 12 so that the first stopping means 10 of the cartridge and the obstructions 19, 20 of the housing 12 are out of alignment, which means the first stopping means 10 and the first and second obstructions 19, 20 would not interact if the cartridge moves axially with respect to the housing 12. The cartridge is positioned in the first positioning during delivery. The distal movement of the cartridge would not be obstructed by the first and second obstructions 19, 20. In a second positioning the cartridge is positioned relative to the housing 12 so that the first stopping means 10 of the cartridge and the obstructions 19, 20 of the housing 12 are in alignment, which means the first stopping means 10 and the first and second obstructions 19, 20 would interact if the cartridge moves axially. When the piston 15 would be retracted the first stopping means 10 and the obstructions 19, 20 would interact as described below.

FIG. 6 shows that the relative position of the cartridge and the housing 12 has changed, e.g. by rotation, before the piston element 15 is retractable. The cartridge has rotated in the housing 12 so that the first stopping means 10 and the first and second obstructions 19, 20 are in alignment.

FIG. 6 shows that the piston 15 is moved in the proximal direction. The cartridge including the needle shield 2 and the body element 5 is retracted in the proximal direction until the stopping means 10 of the needle shield engages with the first obstructions 19, when the piston element 15 is pulled in the proximal direction. The proximal movement of the needle shield 2 is stopped when the needle shield 2 engages with the first obstruction 19. The body element 1 including the needle assembly 5, 7 continues to move in the proximal direction with respect to needle shield 2 when the piston element 15 is further moved in the proximal direction with respect to the housing 12. The resulting relative axial movement of the needle shield 2 and the body element 1 results in retracting the needle 7 into the needle shield 2.

The body element 1 and the needle assembly 5, 7 are retracted with respect to the needle shield 2 to a proximal position. The proximal movement can be stopped by stopping means (not shown) located on the inside wall of the needle shield 2.

After retraction of the body element 1 and the needle assembly 5, 7 to the proximal position, further pulling of the piston element 15 in the proximal direction results in decoupling the first stopping means 10 and the first obstruction 19 if the pulling force is sufficient to decouple. In this embodiment the first stopping means 10 slides over the first obstruction 19 if the pulling force is sufficient.

The cartridge (including the body element 1 and the needle shield 2) is then pulled further in the proximal direction with respect to the housing 12 until the first stopping means 10 reaches the second obstruction 20. The second obstruction 20 may be designed as bumps or elevations.

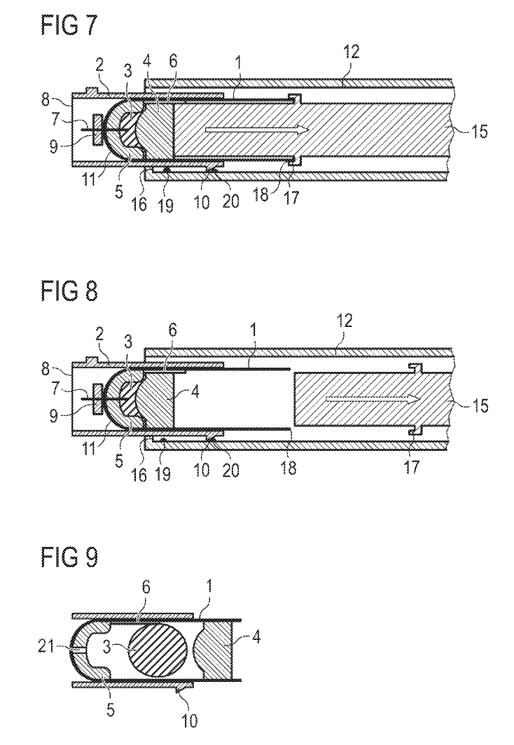

FIG. 7 shows the cartridge inside the medication delivery device, the stopping means 10 of the cartridge having reached the second obstruction 20.

The second obstruction 20 provides sufficient resistance to detach the piston element 15 from the cartridge when the piston element 15 is pulled further in the proximal direction.

In a further embodiment the first stopping means 10 cannot slide over the first obstruction 19. In this further embodiment the second obstruction 20 is unnecessary. The first obstruction 19 provides sufficient resistance to detach the piston element 15 from the cartridge when the piston element 15 is pulled further in the proximal direction.

FIG. 8 shows the cartridge and the detached piston element 15. The cartridge is then removed from the medication delivery device.

The following aspects relate to medication delivery device as described above and a cartridge having a needle shield 2 which is fixed to the body element 1 according to the second embodiment of the needle shield (not shown).

The piston element 15 is releasably coupled with the bung element 4, e.g. by snapping means, in order to retract the needle 7. The dispensing means 5 is attached to the bung element 4 after medication delivery due to an attachment force. In one embodiment the attachment force results from the collapsed capsule 3 located between the dispensing means 5 and the bung element 4. In another embodiment the attachment force results from engaging means configured to engage the bung element 4 and the dispensing means 5, the engaging means engaging when the bung element 4 reaches its furthest distal position.

The cartridge is retracted in the proximal direction when the piston element 15 is pulled in the proximal direction after medication delivery. The proximal movement of the cartridge is stopped when a first stopping means 10 of the cartridge reaches a first obstruction 19.

When the bung element 4 is moved in the proximal direction with respect to the body element 1 due to proximal movement of the piston element 15 with respect to the housing 12, the dispensing means 5 is retracted in the proximal direction with respect to the body element 1. Thus, the needle 7 is retracted into the needle shield 2. In one embodiment the seal 9 which the needle has pierced is removed from the distal wall 8 of the cartridge when the needle 7 is retracted.

The dispensing means 5 is retracted to a proximal position within the needle shield 2. One embodiment of the needle shield 2 is configured to stop the proximal movement of the dispensing means 5. The proximal movement can be stopped by stopping means located on the inside wall of the needle shield 2 or by friction between the dispensing means 5 and the needle shield 2.

After retracting the bung element 4 to the proximal position within the cartridge, further pulling of the piston element 15 in the proximal direction with respect to the housing 12 results in decoupling the first stopping means 10 from the first obstruction 19 if the pulling force is sufficient to decouple. In one embodiment the first stopping means 10 slides over the first obstruction 19 if the pulling force is sufficient.

The cartridge is then pulled further in the proximal direction until the first stopping means 10 reaches the second obstruction 20. The second obstruction 20 provides sufficient resistance to detach the piston element 15 from the cartridge when the piston element 15 is pulled in the distal direction. Then the cartridge can be removed from the medication delivery device.

In an alternative embodiment (not shown) one obstruction is provided which is configured to engage with the first stopping means when the cartridge is moved in the proximal direction. When the piston element 15 is pulled in the proximal direction, the obstructions provide sufficient resistance that the dispensing means 5 is retracted with respect to the needle shield 2 until the dispensing means 5 reaches its furthest proximal position, then the piston element 15 is detached from the cartridge. The obstruction provides sufficient resistance to detach the piston element 15 from the cartridge when the piston element 15 is pulled further in the distal direction.

In another embodiment only the bung element 4, dispensing means 5 and needle 7 are moved by the piston element 15 in the proximal direction and the cartridge does not move at all with respect to the housing 12.

One embodiment of the cartridge (not shown) is used in conjunction with a pump delivery system. The pump draws the fluid from the capsule and dose accurately. The pump delivery system sucks the drug out of the capsule and dispenses the product. The capsule and the needle placement facilitate the effective use of such a pump.

FIG. 9 shows an alternative embodiment of a cartridge suitable for a needle-less medication delivery system.

The cartridge for a medication delivery device comprises a body element 1, immoveable dispensing means 5 and a bung element 4. A deformable capsule 3 containing medication is located inside a chamber formed by the body element 1, the dispensing means 5 and the bung element 4. The capsule 3 has a flexible skin which encases the medication. The capsule 3 contains a single dose of the medication to be dispensed.

The dispensing means 5 comprising a jet spraying nozzle 21 is configured to deliver the medication in a jet spraying manner. As the bung element 4 is pushed distally the capsule 3 is pushed against the dispensing means 5 so that the capsule 3 is broken. The pressure of a medication delivery device ruptures the capsule 3 so that the medication to be dispensed is provided. The medication is injected into or distributed over a region of the body through the jet spraying nozzle 21.

The cartridges for needle-less embodiments as well as for embodiments having a needle can e.g. be used in rapid vaccination processes such as immunization. The speed of administering the medication is increased in comparison with the conventional vaccination process, because there is no need to dial a dose and/or to replace a needle.

Other implementations are within the scope of the claims. Elements of different embodiments may be combined to form implementations not specifically described herein.

### REFERENCE NUMERALS

1 body element
2 needle shield
3 capsule
4 bung element
5 dispensing means
6 gas exhaust
7 needle
8 distal wall
9 seal
10 first stopping means
11 block element
12 housing
13 drive element
14 button part
15 piston element
16 stopping edge
17 second stopping means
18 distal end of capsule
19 first obstruction
20 second obstruction
21 jet spraying nozzle

1. Single-use cartridge for a medication delivery device, comprising
a body element having a proximal end and a distal end,
a dispenser, and
a deformable capsule containing medication, the capsule being disposed inside the body element and having a skin which encases the medication, the capsule being configured to be pushed in the distal direction against the dispensing means so that the content of the capsule is dispensed.

2. Cartridge according to claim 1, further comprising a bung element which is moveable in the distal direction with respect to the body element upon being pressed by external force and which is provided for pushing the capsule against the dispensing means.

3. Cartridge according to claim 1, wherein the dispense is configured to puncture or rupture the capsule.

4. Cartridge according to claim 1, wherein the dispenser comprises an injection needle which comprises a proximal end configured to puncture the capsule.

5. Cartridge according to claim 1, wherein the dispenser comprises a gas exhaust configured such that gas escapes when the capsule is pushed towards the dispensing means.

6. Cartridge according to claim 1, wherein the dispenser is moveable in an axial direction with respect to the body element.

7. Cartridge according to claim 1, further comprising a needle shield the needle is disposed in.

8. Cartridge according to claim 7, wherein the needle shield is moveable in the distal direction with respect to the body element.

9. Cartridge according to claim 7, wherein the needle is moveable in the distal direction with respect to the needle shield so that a distal part of the needle extends out of the needle shield.

10. Cartridge according to claim 7, wherein a distal wall of the needle shield comprises a seal or wherein a distal wall of the body element comprises a seal, the seal being configured to be punctured by the needle when the needle is moved in the distal direction.

11. Cartridge according to claim 7, wherein the needle is moveable in the proximal direction with respect to the needle shield after moving in the distal direction so that the needle retracts into the needle shield.

12. Cartridge according to claim 7, wherein the dispenser and the capsule after medication delivery are moveable in the proximal direction with respect to the needle shield when the bung element is moved in the proximal direction with respect to the needle shield.

13. Cartridge according to claim 1, wherein the dispenser and the capsule after medication delivery are moveable in the proximal direction with respect to the body element when the bung element is moved in the proximal direction with respect to the body element.

14. Cartridge according to claim 1, comprising first coupling means releasably coupleable with a piston element of a medication delivery device.

15. Cartridge according to any of the claims 1, wherein the dispenser is configured to deliver the medication in a jet spraying manner.

16. Medication delivery device comprising the cartridge according to claim and a housing, wherein the delivery device is configured to move the capsule and/or the bung element in the distal direction.

17. Medication delivery device according to claim 16, comprising a piston element moveable in the distal direction with respect to the housing.

18. Medication delivery device according to claim 17, wherein the piston element is retractable in the proximal direction with respect to the housing.

19. Medication delivery device according to claim 17, wherein the piston element comprises a second coupling means releasably coupleable with a first coupling means of the cartridge.

20. Medication delivery device according to claim 18, wherein the delivery device comprises obstruction means configured to stop or prevent a proximal movement of the cartridge with respect to the housing when the piston element is moved in the proximal direction with respect to the housing so that the piston element can be detached from the cartridge
